(19) United States
(12) Patent Application Publication
(10) Pub. No.: US 2021/0123818 A1
(43) Pub. Date: Apr. 29, 2021

(54) THERMAL SENSING WITH BLACKBODY RADIATION

(71) Applicant: Miami University, Oxford, OH (US)

(72) Inventor: Hui Wang, Oxford, OH (US)

(21) Appl. No.: 17/082,695

(22) Filed: Oct. 28, 2020

Related U.S. Application Data
(60) Provisional application No. 63/007,590, filed on Apr. 9, 2020.
(60) Provisional application No. 62/926,853, filed on Oct. 28, 2019.

(51) Int. Cl. G01K 11/32; A61B 18/28; A61B 5/01; A61N 7/02; A61B 90/00

(52) U.S. Cl. CPC G01K 11/32; A61B 18/28; A61B 2018/2211; A61N 7/02; A61B 90/37; A61B 5/01

(57) ABSTRACT

A method and apparatus using radiation-based fiber-optic sensors and ultrasound thermometry to detect temperature before and during surgery. Ultrasound thermometry accurately measures temperature less than 50° C. and requires calibration, which can be conducted in vivo with the disclosed fiber sensor based on blackbody radiation (BBR) and as an early step in the procedure. The monitored wavelength of BBR in a range between about 1.4 μm and about 2.7 μm results in low attenuation for both water and a silica-based fiber. A thermal boundary map at and around the boundaries of the subsequently heated tissue in the region of interest (ROI) is displayed to the surgeon. The system accurately displays the temperature(s) in a thermal boundary map, thereby permitting the surgeon to determine when the ROI has been exposed to sufficient thermal energy to destroy it.

### CROSS-REFERENCES TO RELATED APPLICATIONS

This application claims the benefit of U.S. Provisional Application No. 62/926,853 filed Oct. 28, 2019 and U.S. Provisional Application No. 63/007,590 filed Apr. 9, 2020.

### STATEMENT REGARDING FEDERALLY-SPONSORED RESEARCH AND DEVELOPMENT

(Not Applicable)

### THE NAMES OF THE PARTIES TO A JOINT RESEARCH AGREEMENT

(Not Applicable)

### REFERENCE TO AN APPENDIX

(Not Applicable)

### BACKGROUND OF THE INVENTION

This invention relates generally to temperature measurement and more specifically to temperature measurement using blackbody radiation for realizing ultrasonic thermometry and others.

During many medical procedures, it is important to monitor the tissue temperature inside of the human body. Blackbody radiation (BBR) between 3 μm to 10 μm has long been used for temperature sensing, but not successfully in medical applications, such as endoscopic applications.

A variety of sensors have been tested for temperature measurement using BBR. Among them, fiber-optic temperature sensors have many unique features, such as flexibility, complete immunity to interference from radio frequency (RF) and microwave radiation, and intrinsic reliability in harsh and corrosive environments. In general, fiber-optic temperature sensors can be categorized as structure-based, material-based, or radiation-based sensors.

Fiber Bragg Grating (FBG) sensors are popular structure-based, fiber-optic temperature sensors. FBG sensors are made by fabricating a fine volume grating in a fiber and detecting temperature-related spectral shift. However, FBG sensors are sensitive to strain and pressure induced by the motion of the human body, such as respiratory movements, making clinical use challenging.

FLUOROPTIC® brand sensors are an example of material-based fiber-optic temperature sensors. The sensors are made by adding a fluorescent material to the tip of a fiber and detecting temperature by measuring temperature-induced fluorescence lifetime decay, spectral shift, or the intensity ratio from two different emission bands. However, such sensors suffer from artifacts due to self-heating when used during laser thermotherapy.

Pyrometer fiber sensors, a radiation-based temperature sensor, can measure the black body radiation emitted from very hot surfaces (>300° C.). They are usually used in extremely harsh environments that other sensors cannot access. For detecting temperatures lower than 100° C., pyrometer fiber sensors often require special infrared fibers to transfer BBR at midrange infrared wavelengths (MIR, ˜3 μm-˜8 μm). In principal, a radiation-based fiber-optic sensor is very attractive because it does not require physical contact with tissues. This is because BBR can be detected even if there is a gap between the fiber-optic sensor and the tissue surface. However, silica fiber, which is popularly used to build fiber catheters, cannot transmit BBR in MIR.

Fiber-based temperature sensing is very useful in areas that are difficult to access, but the temperature-related blackbody radiation must be transferred through a few meters of optical fiber. Because the attenuation caused by the fiber of a signal in the wavelength range of 3 μm to 10 μm is very high, detection of BBR in this wavelength range is not feasible using conventional methods and apparatuses.

Further complicating matters, saline is typically used to cool down a surgical area during thermal (e.g., laser) surgery. The absorption of water in the frequency range of 3 μm to 10 μm can completely attenuate blackbody radiation in a few tenths of micrometers of water thickness. Although there are patents claiming to use fibers and blackbody radiation for temperature monitoring, none of them can work in the surgical environment. Examples of these include U.S. Pat. No. 4,576,486 to Dils and U.S. Pat. No. 4,845,647 to Dils et al.

In some surgical treatments, a surgeon applies thermal energy to living tissue, which may be a tumor or another isolated tissue in the human body. The purpose of the application of thermal energy is to damage the tissue, and, in the case of a tumor, to completely destroy the harmful tumor tissue so that it poses minimal subsequent harm to the person. Thermal energy may be applied by a laser or any other surgical instrument to heat this tissue to a temperature for a period of time at which the tissue cannot survive. Calibration of the instrument to ensure destruction of the tissue is conventionally performed prior to the surgery using animal tissue or some other means. The obtained calibration curve causes some error during surgery since the calibration is on ex vivo tissue that is not the same tissue being treated by the surgeon.

The need exists for means and methods for accurately detecting temperature during thermal and other surgeries.

### BRIEF SUMMARY OF THE INVENTION

Disclosed herein are methods and apparatuses using radiation-based fiber-optic sensors and ultrasound thermometry to detect temperature before and during surgery. These methods and apparatuses may limit the monitored wavelength of blackbody radiation (BBR) to a range between about 1.4 μm and about 2.7 μm. This range has relatively low attenuation for both water and silica-based fiber. The measurement may be through a fiber catheter, used before or during laser surgery, but is not limited to this. Temperature measurement using BBR is for the purpose of calibrating the ultrasonic temperature measuring device, which may accurately monitor temperature during the surgery. Using the ultrasonic thermometry equipment to detect the temperature at the boundaries of the region of interest (ROI) at the surgical site, which may be a tumor, the system accurately displays to the surgeon the temperature at the boundaries of the ROI, thereby permitting the surgeon to determine when the ROI has been exposed to sufficient thermal energy to destroy the ROI.

A method is disclosed for monitoring temperature with blackbody radiation (BBR) in a wavelength range between 1.4 μm and 2.7 μm, as shown generally in the illustration of FIG. 1. The FIG. 1 flow chart refers to several specific methods and apparatuses, which are disclosed herein to monitor the temperature based on blackbody radiation. Regarding the illustration of FIG. 1, any detector can be used as long as it can detect BBR in a range of about 1.4 μm to about 2.7 μm. Such a detector could be a single or an array detector.

It is preferred to specify the wavelength range of about 1.4 μm to about 2.7 μm because of low attenuation of fiber and relatively low water absorption in this range, as shown in the graph of FIG. 2. Preliminary data has been acquired in support that this method can detect temperature down to less than 40° C.

FIG. 3 shows the results of a fiber sensor that was calibrated three times with a standard BBR source. The mean values, which are shown in FIG. 3 by the indicator “x”, were fitted with a power function. A water layer with a thickness of 0.5 mm was placed ahead of the fiber end and the measured signals, which are shown by squares, were plotted in FIG. 3.

FIG. 4 shows a graphical representation of blackbody radiation measured with the fiber sensor and temperature simultaneously with a thermocouple by inserting both into chicken muscle to simulate laser interstitial thermal therapy. The measured blackbody radiation is converted to temperature with the calibration equation obtained in FIG. 4.

Disclosed herein is a method and apparatus to calibrate ultrasound thermometry equipment using a BBR thermal sensor. Ultrasound thermometry can only linearly respond to temperature less than 50° C. and requires calibration, which is tissue dependent. The calibration can be conducted in vivo with the fiber sensor based on BBR and preferably as an early step in the surgical treatment, and then the surgeon can track the development of thermal boundary maps less than 50° C. around the subsequently heated tissue. The thermal boundary map provides the surgeon an intuitive means for tracking the development of the heated region, thereby permitting the surgeon to determine when the heated region has been sufficiently heated. The apparatus and method may be used on any living tissue, including that of humans and animals.

### BRIEF DESCRIPTION OF THE SEVERAL VIEWS OF THE DRAWINGS

FIG. 1 is a flow chart illustrating an embodiment of using black body radiation from tissue to detect temperature.

FIG. 2 is a graphical illustration of a simulation of a BBR signal after passing through a 2 meter silica fiber and 0.5 mm of water.

FIG. 3 is a graphical illustration of the measured temperatures calibrated with a standard black body radiation light source.

FIG. 4 is a graphical illustration of temperature using various means.

FIG. 5 is a schematic view illustrating two examples of multiple core fiber.

FIG. 6 is a graphical illustration of wavelength versus frequency for water extinction length and molecular extinction coefficient.

FIG. 7 is a schematic illustration showing an apparatus for detecting BBR at different wavelengths.

FIG. 8 is a graphical illustration of BBR at different temperatures.

FIG. 9 is a graphical illustration of water and silica fiber absorption.

FIG. 10 is a schematic illustration of a simulation of BBR signals from different depths of tissue for the purpose of temperature measurement.

FIG. 11 is a schematic illustration of modulated laser power through a fiber.

FIG. 12 is a schematic illustration of a flow chart of a process for generating a virtual heat map.

FIG. 13 is a schematic illustration of a thermal boundary map reconstructed by combining an ultrasonic thermometer and a BBR thermal sensor.

FIG. 14 is a schematic illustration of a thermal boundary map reconstructed by combining an ultrasonic thermometer and a BBR thermal sensor.

FIG. 15 is a schematic illustration of an apparatus for gap detection.

In describing the preferred embodiment of the invention, which is illustrated in the drawings, specific terminology will be resorted to for the sake of clarity. However, it is not intended that the invention be limited to the specific term so selected and it is to be understood that each specific term includes all technical equivalents which operate in a similar manner to accomplish a similar purpose. For example, the word connected or terms similar thereto are often used. They are not limited to direct connection, but include connection through other elements where such connection is recognized as being equivalent by those skilled in the art.

### DETAILED DESCRIPTION OF THE INVENTION

U.S. Provisional Application No. 62/926,853 filed Oct. 28, 2019 and U.S. Provisional Application No. 63/007,590 filed Apr. 9, 2020, which are the above prior applications, are hereby incorporated in this application by reference.

Disclosed herein are methods and apparatuses for detecting a temperature field during surgery on human or animal tissue so that harmful tissue may be destroyed and non-harmful tissue is preserved as much as is feasible in view of the interest in destroying adjacent harmful tissue. In one embodiment, ultrasonic thermometry is used to measure the temperature of the tissue during at least some portion of the procedure, which is typically the latter portion, and the ultrasonic equipment may be calibrated for accuracy using BBR detecting methods and equipment.

The calibration according to the invention is performed during the surgery, preferably in the earlier portion, although calibration in a later portion is contemplated. Indeed, multiple calibrations throughout surgery is contemplated. The calibration may be performed using a BBR sensor, such as any of the BBR sensors described herein, and the methods disclosed herein or any conventional method. The BBR sensor may be limited to detecting BBR at wavelengths in a range between about 1.4 and about 2.7 μm.

The embodiment uses BBR thermal sensing to calibrate the ultrasonic thermometry equipment, thereby resulting in a high degree of accuracy when using the ultrasonic thermometry equipment during the subsequent portions of the surgical procedure. In one embodiment, the ultrasonic thermometry equipment is used to create visual images, which may be images that indicate temperature using particular colors, patterns, textures or other visual indicators for particular temperatures. For example, an image may contain colors that are considered “warm” (e.g., red, orange, yellow, etc.) to designate warmer temperature areas and colors that are considered “cool” (e.g., violet, indigo, blue, etc.) to designate cooler temperature areas. Examples of an image with colored regions are shown in FIGS. 13 and 14. These images may be used by the surgeon to determine when sufficient thermal energy has been applied in particular regions.

A thermal boundary map is formed during surgery or other treatment by combining the ultrasound thermometer and local temperature measured with a thermal sensor using blackbody radiation. Ultrasound thermometry is a convenient and inexpensive way to generate the desired thermal map. Ultrasonic thermometry is based on the relationship between the velocity of ultrasound and the properties of the medium the ultrasound travels through. The temperature along the travel path can be calculated after measuring the ultrasound velocity between an ultrasonic transmitter and a receiver. The ultrasonic speed is determined by the distance the ultrasound travels and the ultrasonic time-of-flight (UTOF). The medium composition and the distance must be obtained to calculate ultrasonic speed, and the temperature can be inferred from the UTOF. The accurate measurement of the UTOF is the key for ultrasonic thermometry, and the calibration determines the UTOF.

The tissue temperature change estimated by echo-shifts is known. Two thermal-dependent parameters induce echo-shifts: the thermal dependence of the speed of sound (SOS) and thermally induced physical expansion of the tissue sample. Although echo-shifts based thermometry can principally track temperature change, the quantification requires prior knowledge of the linear coefficient of thermal expansion, a, and tissue-dependence of the change of SOS with temperature, β. The temperature change can then be quantified using the equation

$$Δ T ( z ) = \frac{{c}_{0}}{2 ( α - β )} \frac{δ t ( z )}{δ ( z )} ,$$

where t(z) is the measured echo-shift at depth z and c₀ is the SOS before heating tissue. The term c₀/2(α−β) highly depends on tissue type, such as fat content, and conventionally needs to be determined by ex vivo calibration. This is problematic in clinics because the tissue types between patients are often different. For employing ultrasound thermometry in clinics, developing in vivo calibration technology is an important step. However, the technology for in vivo calibration has not been available, possibly because inserting an extra thermal sensor, and thereby forming another tissue opening, is not usually acceptable during surgery. In addition, the linear relation between the echo-shift and the temperature change is only accurate up to temperatures in the range of 45-55° C. Therefore, ultrasound thermometry has been proposed to monitor only hyperthermia, which is less than about 50° C., not ablation (which occurs above 50° C.) that is the subject of thermal treatments that destroy living tissue.

A contemplated method includes a step of first calibrating an ultrasound thermometer, which can be conducted in vivo during the surgical procedure using a BBR thermal sensor, and then tracking the development of thermal boundary maps less than about 50° C. around the heated tissue using ultrasound thermometry. The thermal boundary map may be used by a surgeon to track the development of the heat region, as part of the process. The surgical process may be the destruction of a tumor (the ROI) by imparting thermal energy to the tumor using a laser or another instrument.

As shown in FIG. 13 before laser ablation, a region of interest (ROI) can be identified through pre-surgery imaging, such as by using MRI or CT imaging. During ablation (FIG. 14), surgery is guided under live ultrasound thermal imaging. The ultrasound images are fused with the MRI images to delineate the ROI and guide the insertion of the fiber catheter. At the start of the surgical procedure, and optionally at the start of ablation, tissue-dependent thermal parameters for ultrasound thermometry are calibrated using the BBR fiber temperature sensor as the reference. The expansion of the thermal boundary map, which is usually the portion that is less than 50° C., during heating is tracked using the echo-shifts of the ultrasound images, shown as the blue region in FIG. 13. Thermal boundary maps can provide intuitive guidance to surgeons to track whether the tissue around the margins of the ROI has been effectively treated. The region between the fiber tip and the boundary of the ROI can be numerically interpolated by assuming the temperature distribution is continuous and smooth. Alternatively, a simulation may be used to determine the temperatures between the fiber tip and the thermal boundary. This is explained further herein.

In order to perform in vivo calibration, a temperature sensor must be inserted into the surgical site. In at least one embodiment, a single instrument is used as a thermal sensor to calibrate the ultrasonic thermometry equipment and as the structure through which is performed the surgical procedure, such as a catheter. Thus, only one opening in the patient may be needed to perform the entire procedure—the calibration and the thermal destruction of the ROI. It is possible to calibrate the ultrasound equipment, create the thermal boundary map, and send energy to the tumor or other ROI through a single instrument, which may be a catheter, and may include an optical fiber through which a laser imparts thermal energy to the ROI and through which BBR is detected. The catheter may be another type as described herein or known to the person of ordinary skill as equivalent.

In the procedure, a single opening is formed in the tissue through which the instrument is extended. After the instrument is inserted into the patient's tissue, a BBR thermal sensor performs the in vivo calibration to obtain a calibration curve specific to the tissue. This calibration step may be at the same time that ablation by the same instrument is taking place. The obtained curve data used during the surgery on the same tissue, typically without removing the instrument from the patient. A single catheter is thus used to perform the calibration and the surgical procedure of imparting thermal energy to the ROI. During at least some portion of the procedure, and possibly during most or all of the procedure, ultrasound images are used to convey to the surgeon the temperature distribution around the ROI.

The surgeon may view one or more displayed images that communicate temperature in different regions of the tissue, and at least at the regions local to the ROI. The images are obtained using ultrasonic thermometry to measure the temperature distribution around the boundaries of the region of interest (ROI), which may be a tumor. As noted above, there are limitations with ultrasound sensors, and they are conventionally considered accurate when measuring temperatures up to 50° C. Above 50° C. ultrasound thermometry is not accurate. Therefore, the ultrasonic equipment measures the temperature at the boundaries around the ROI, which may be about, or below, 50° C.

Using the measured temperatures, the system creates an accurate thermal boundary “map,” which is an image that communicates to a human surgeon the temperature(s) at and/or around the boundaries of the ROI. The map may show the temperatures in the ROI and all visible surrounding regions. Alternatively, the map may show the temperatures in all visible regions surrounding the ROI defined by the surgeon or someone else, such as within 20 percent of boundaries of the largest dimension of the ROI. Alternatively, the map may show the temperatures within 5 centimeters of the boundaries of the ROI: both within and outside of the ROI. Regardless of the portion displayed to the surgeon, the surgeon is shown a map with accurate temperature indications at least at and/or adjacent to the boundaries of the ROI so that the surgeon is able to determine when the ROI has been exposed to sufficient thermal energy to destroy the ROI, and also to prevent the loss of more of the tissue surrounding the ROI than necessary.

The location of the ROI may be determined before or during the surgical procedure by an MRI, a CT scan or any other imaging equipment or method. After this determination, the image(s) created by the ultrasonic thermometry equipment may be combined with the image(s) created by the MRI or CT scan to create an image that conveys to a surgeon the temperatures and the boundaries of the ROI. This may be accomplished by taking one image and placing it over the other image. If the backgrounds are transparent, the data for both will remain visible after overlapping. It is also contemplated that software may be developed that integrates the images and/or the data created by the different technologies so much that the two images are not discernible from one another. This may result in a single image displaying data from both devices. Furthermore, the images may be displayed in such a manner that the surgeon is able to manipulate the images (e.g., rotate, pan, magnify, and otherwise alter the image visible to him or her on a screen or other display) and the boundaries of the ROI obtained from the MRI or CT scan maintain their relative position to the thermal images obtained by the ultrasonic thermometry equipment during this manipulation. The display may be a screen, goggles, microscope lens, or any other human-perceptible visual display.

In some embodiments, the MRI images integrate with the ultrasonic thermometry-produced images so well that the surgeon does not readily distinguish between them. Instead, a single image is seen on one display, and that single image includes the accurate location of the boundaries of the ROI from the MRI, and the accurate colors created by the ultrasonic thermometry equipment that convey information about the temperature at and near the boundaries of the ROI. This “thermal map” enables the surgeon to administer sufficient thermal energy to the ROI, and perhaps some of the surrounding area, to destroy the tissue of the ROI while preserving as much of the surrounding tissue as he or she deems desirable. A selected amount of tissue surrounding the ROI may also be subject to a temperature that is damaging to ensure that the ROI tissue is sufficiently heated to be destroyed, and this is determined by the surgeon as informed by the temperature information presented on the display.

The temperature distribution communicated in the thermal map has a temperature gradient. For example, with a tumor one may measure temperature only at the boundary and the surrounding regions instead of at the center of the tumor where it is higher than 50° C. and the temperature may not be measured accurately. By sensing temperature at the boundary, where the temperature measurement by ultrasonic thermometry is accurate, it can be confirmed that the temperature inside of the ROI reaches a temperature sufficiently higher than at the boundaries due to the thermal energy being imparted to the patient at a point inside the ROI. Thus, this destroys the ROI and the temperature outside of the ROI does not reach a temperature higher than a predetermined maximum, which may be 50° C., for a period of time sufficient to destroy the tissue. As long as the destructive apparatus, such as a laser, can supply the ROI with enough thermal “doses,” the surgeon can surmise that the ROI is destroyed using only the temperature measurement at the margins/boundaries of the ROI. Thus, viewing of the thermal temperature boundary map is very important.

In one embodiment, after the calibration, the surgeon measures temperatures of the tissue only at the boundaries of the ROI where the temperature is at or less than 50° C. Even though the surgeon may not be able to determine the temperature in the ROI due to inaccuracies inherent in ultrasonic thermometry, the surgeon is aware of the temperature(s) at the boundaries. And if the surgeon knows that the temperatures inside the boundaries are higher (although they are not known with accuracy), then the surgeon may reasonably conclude when the ROI has received a sufficient dose of thermal energy to make destruction of the tissue in the ROI all but certain. Thus, the system uses knowledge of the temperatures at the boundaries to determine whether the ROI has been exposed to sufficient thermal energy for a sufficient period to destroy the ROI.

The temperature is dynamic, for example due to blood vessels removing thermal energy, and if the temperature is close to 50° C. at the boundaries of the ROI, then the surgeon can determine whether and when the temperature is sufficiently high in the ROI to destroy the tissue of the ROI. The surgeon uses the apparatus described herein to measure temperature accurately at least at the boundaries of the ROI to make sure the entire ROI has been destroyed. The surgeon may also expose healthy or non-harmful tissue (surrounding the ROI boundaries) to sufficient thermal energy to ensure that the ROI is destroyed, even if exposing that healthy tissue results in the destruction of some of that healthy tissue. The objective is to create a margin, even if some healthy tissue is damaged, to ensure that all of the tissue in the ROI is destroyed.

In one embodiment, a first step is to use magnetic resonance imaging (MRI), computerized tomography (CT) scan or other means to create a human-perceptible image showing the precise boundaries of the tumor or other ROI. Next, the boundary image of the MRI or other technology is combined with the ultrasound image created during surgery that indicates temperature in some or all regions thereof. The combining of images and/or data that creates the images is known in the industry, as evidenced by a paper titled “Image Fusion Using CT, MRI and PET For Treatment Planning, Navigation And Follow Up In Percutaneous RFA” and published in Exp Oncol. 2009 June; 31(2): 106-114 as well as a web page at http://surgery.ucla.edu/prostate-cancer-diagnosis-via-ultrasound-mri-fusion, both of which are incorporated herein by reference. The combining of images may be a continuous process in which temperature-conveying information, or an image with that temperature-conveying information, is updated periodically and, optionally, automatically on the display. The surgeon visually monitors the temperature while imparting the thermal energy to the ROI using a laser or other equipment in all areas of concern, thereby monitoring for when the tissue-damaging temperature has spread to, or near or exceeding, the boundaries of the ROI. Once the ROI tissue and any desired surrounding tissue have been heated to a sufficient temperature for a sufficient period, the heating step is halted to prevent or limit the thermal damage to normal/healthy tissue.

The method and apparatus result in a new treatment strategy for laser or other thermal treatment. The method includes using a single fiber to perform in vivo calibration to accurately quantify the temperature curve that represents the characteristics of the tissue. At least some data are collected regarding the precise location of the boundaries of the ROI, preferably using MRI, CT scan or other, and some data are collected regarding the temperature at least near the boundaries of the ROI, preferably using ultrasonic thermometry. The data are combined or “fused,” which results in the combination of the MRI data (indicating the precise location of the ROI boundaries) and the thermal data from the ultrasound into one or more images that are visually perceptible to a human user. Fusing defines the boundaries of the ROI and the ultrasound thermal image that shows temperature gradients and forms a single thermal boundary map the surgeon can use to see the temperatures at least at and/or near the boundaries of the ROI. This permits a surgeon to determine when a desired temperature has been reached at or near the boundary of the ROI. The surgeon is able to determine, from this display, when all ROI tissue inside the boundary is at a higher, and more destructive, temperature after the fiber is inserted in the ROI and thermal energy is imparted to the ROI. If the temperature at the boundary has been about 50° C. for a sufficient period while heating up the ROI with the fiber, then one can conclude that the ROI tissue has been destroyed.

The fibers used for thermal sensing in any of the herein-described systems and/or processes can be designed to have different forms. Many fiber embodiments are contemplated, including a first embodiment in which a single silica fiber is used for temperature sensing only. In this embodiment, a single fiber is used for the sole purpose of sensing temperature. In another embodiment, a single fiber may be used for both temperature sensing and treatment (or for other purposes), such as conveying the thermal energy, such as by using a laser. For example, a single fiber may be used for both thermal sensing and delivering thermal energy, such as for laser surgery. The fiber may be a normal fiber or it may be a processed fiber (beam-focused, lantern beam or with a fiber cap).

When the temperature measurement is performed, there may be some interference, such as between a laser and the temperature field. A gap detection method may be used when a single fiber is used. In this method, the temperature is detected during the intervals while the laser is off. The treatment laser is modulated, as shown in FIG. 11 in which “on” indicates when tissue ablation occurs with the laser, while “off” indicates when the laser power is reduced or switched off. The BBR signal can be detected during the “off” period to avoid potential interferences from the interaction between the laser and the fiber.

As shown in FIG. 15, laser power for ablation can be modulated externally or internally to turn the laser on and off. The detector for detecting the BBR from the tissue can be triggered on and off reversely to the on and off status of the laser. The detector can also be turned on all the time. Then data when the laser is “on” can be discarded through later data processing. In this way, the laser modulation signal should also be sampled into the processer, which could be a computer.

In another embodiment, a coaxial dual-core structure is shown schematically on the left in FIG. 5. The inner core fiber (core 1) may be used for imaging, delivering therapeutic laser energy, or both. The outer core 2 may be an annular fiber used for thermal sensing. In another embodiment shown schematically on the right of FIG. 5, which is another form of multiple core fiber, core 1 and core 2 are separated and not coaxial, but both cores are still in the same fiber. The cores may be used for the same purpose or different purposes. Core 1 or core 2 can be used for thermal sensing or other purposes, such as imaging or laser ablation, and the other may be used for another purpose. There is no limit to the number of cores that may be used. Therefore, if necessary, multiple cores can be used for different purposes. These cores could be single-mode or multi-mode. The fibers used as cores may be made of silica or doped silica.

The thermal sensing fiber may be integrated or bundled with other catheters. In one embodiment, a thermal sensing fiber may be used with catheters for different purposes, such as a radiofrequency ablation catheter, an ultrasound probe, or an imaging fiber, such as an optical coherence tomography catheter or a fluorescence imaging catheter. It is preferred to use a silica fiber as the sensing fiber due to the BBR wavelength range being measured and the ability of such a fiber to transmit laser energy of the wavelength desired. BBR above about 2.7 μm is not conveyed, or is negatively affected, by a silica fiber or doped silica fiber, but this BBR is not desirably measured or detected in the present invention. Silica fibers or doped silica fibers are therefore inexpensive fibers that are useful for the present invention but not for conventional systems detecting BBR, because conventional systems detect BBR at wavelengths of about 3 μm and higher. Thus, silica fibers and doped silica fibers are a surprising material to use for measuring BBR.

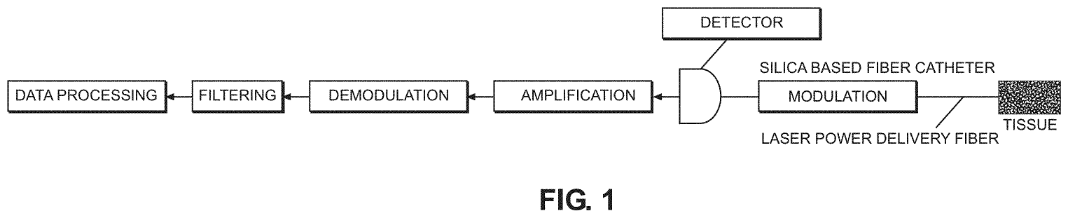

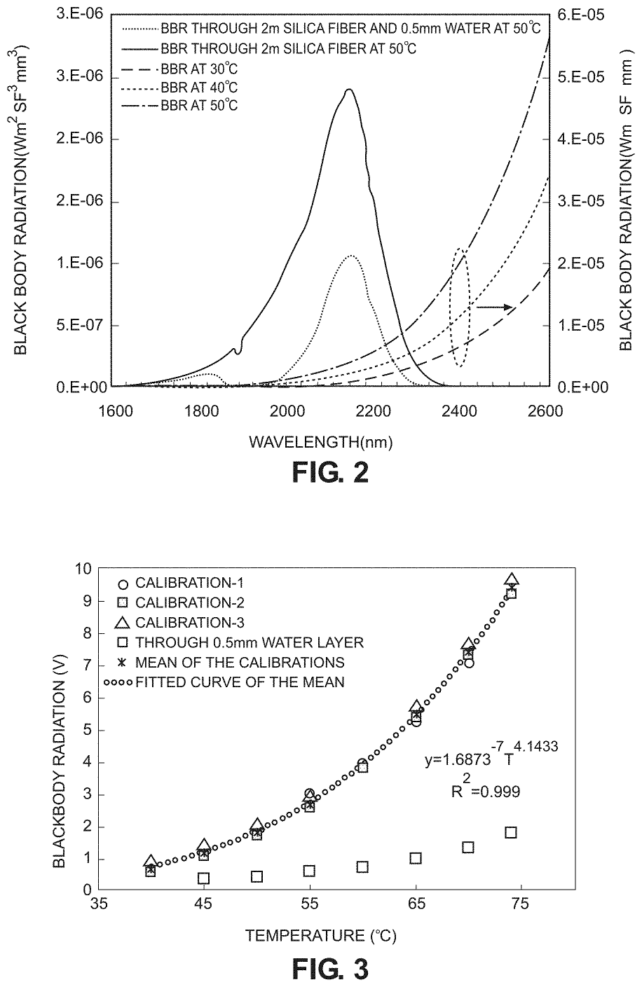

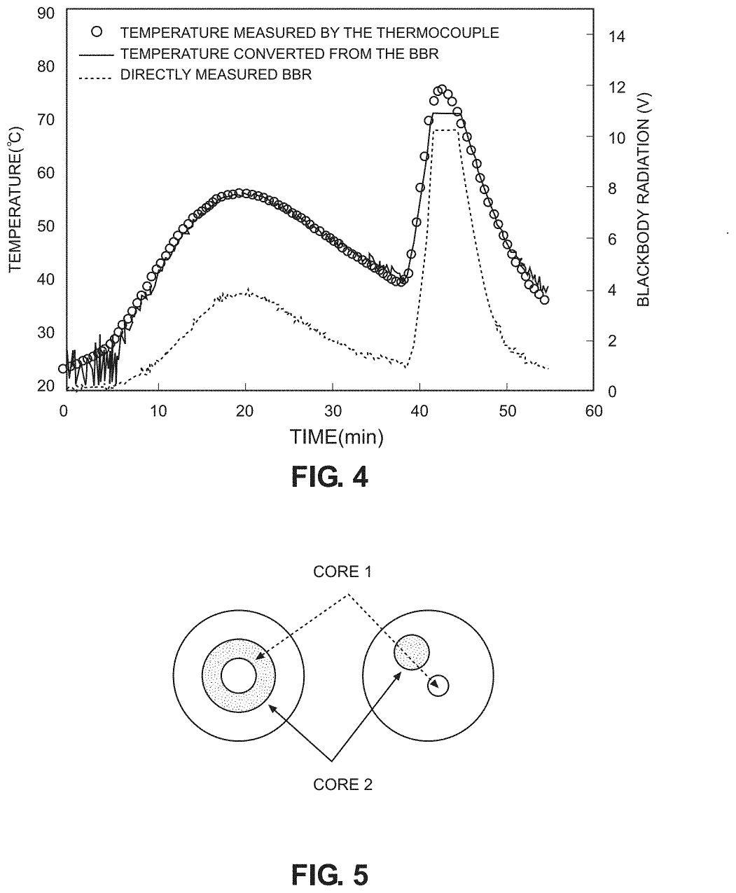

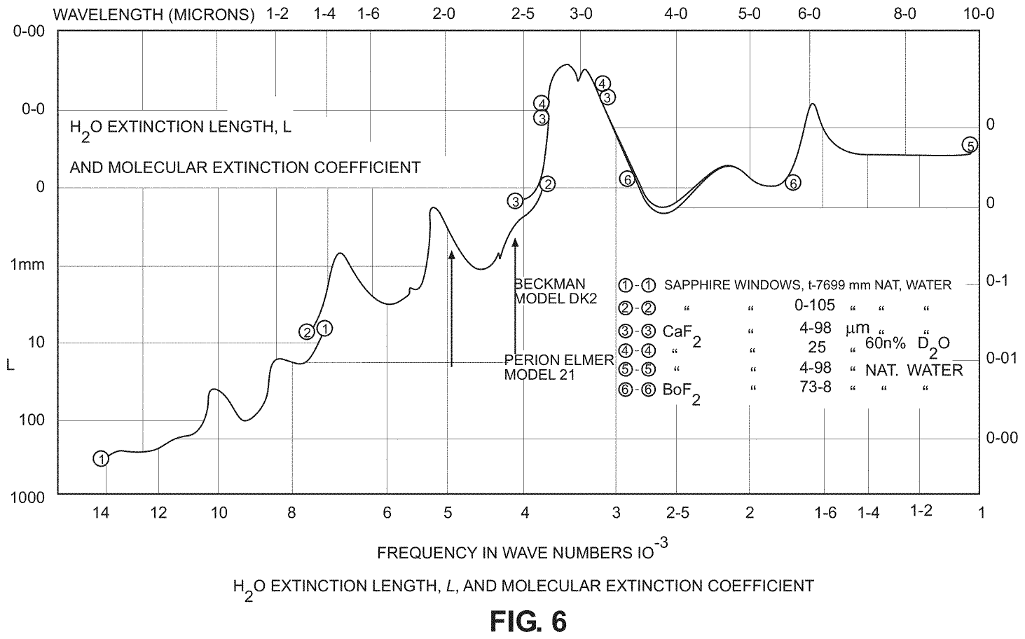

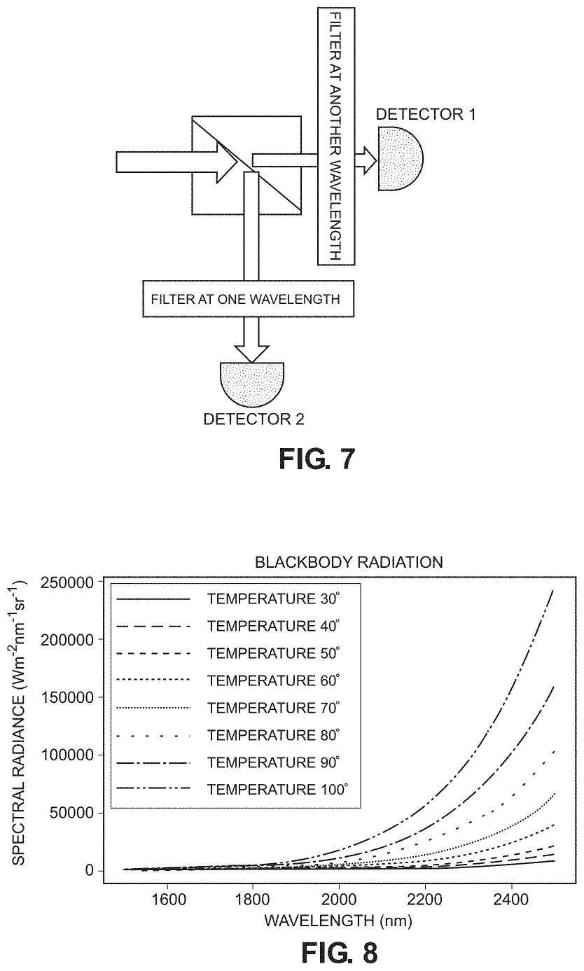

The BBR signal may be affected by the thickness of water or the tissue's optical properties according to Beer's law. This issue can be solved by detecting two wavelengths with the same absorption coefficients or different known absorption coefficients, as shown in the vertical arrows in FIG. 6. After acquiring two signals at two different wavelengths, which could be 2.1 μm and 2.4 μm, one may calculate the ratio of the signals to measure the temperature. The ratio of one wavelength to a different wavelength has a 1:1 relation to a temperature. The layout of the detector portion of FIG. 1 can be modified as shown in FIG. 7. As shown in FIG. 8, the slopes between two different wavelengths are different at different temperatures based on the black body radiation (BBR) calculated from Planck's equation. If the BBR at two different wavelengths can be measured, the absolute temperature can be determined, especially if water absorption is similar at the different wavelengths, such as 2.1 μm and 2.4 μm.

BBR temperature detecting methods currently developed can only monitor temperature from the tissue surface because of strong water absorption in BBR wavelengths between 3 μm and 10 μm. However, BBR is not just from the surface of an object but from the integration of the volume of the object. One of the embodiments disclosed herein uses multiple detectors at different wavelengths to detect surface BBR in the wavelength range between 1.4 μm and 2.7 μm. The BBR is filtered into different wavelengths, or a spectrometer could be used, so the signal of each wavelength may be detected.

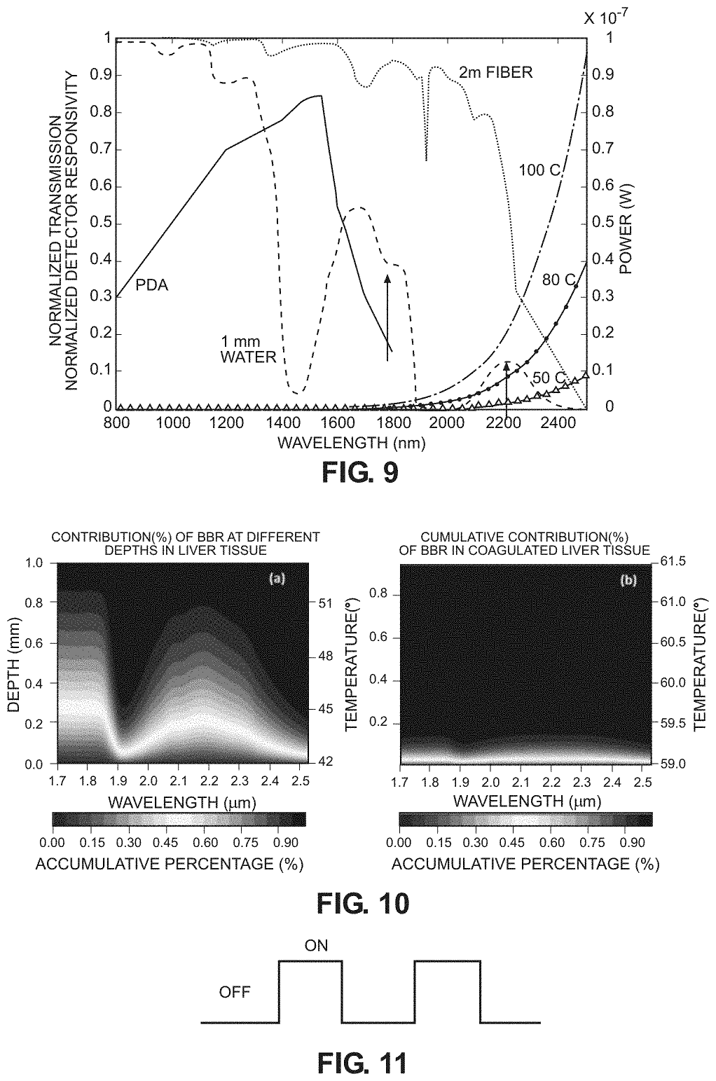

Detection of the surface temperature is accomplished using specific wavelengths at which one may only detect BBR from the superficial surface (I_{BBR surface}), such as at 2 μm and 2.5 μm. Detection of the subsurface temperature is accomplished using specific wavelengths at which one may also detect BBR from the subsurface (I_{BBR subsurface}), such as at 1.8 μm, 2.2 μm, or other wavelengths that can penetrate tissue more due to less absorption. The detected wavelengths for the subsurface detection are represented by the vertical arrows in FIG. 9. The total BBR at these wavelengths includes superficial surface radiation and subsurface radiation from inside the tissue as shown in Equation 1.

I_{BBR total}=I_{BBR surface}+I_{BBR subsurface} (Equation 1)

The superficial (surface) radiation at these wavelengths can be derived based on Planck's equation and the measured temperature from the first detection, and then the subsurface radiation can be calculated by subtracting the superficial surface radiation from the total radiation. In this way, the tissue temperature at a subsurface depth can be derived.

The temperature gradient can also be derived. FIG. 10 slide (a) shows schematically the contribution of BBR from different depths of normal liver tissue. The temperature gradient shown in FIG. 10 slide (a) is produced by assuming the tissue surface is flushed with cooling liquid during heating. It is clear from FIG. 10(a) that most of the BBR signals at wavelengths of about 1.9 μm and about 2.5 μm come from a tissue depth of less than 200 μm, while at wavelength of about 2.2 μm the BBR is from a depth of up to 700 μm.

Another embodiment contemplated is a method of determining the tissue coagulation threshold, which is related to destruction of the tissue. If a surgeon monitors the BBR signal ratio at two different wavelengths (for example at 1.95 μm and 2.2 μm) at different temperatures, it is possible to determine when the tissue is coagulated by when the BBR signal ratio, measured at the wavelengths 1.95 μm and 2.2 μm for all temperatures, reaches a steady state despite temperature change. After coagulation, the ratio ceases to change with changes in temperature. FIG. 10 slide (b) shows the temperature of the liver tissue after it is coagulated. Due to scattering after coagulation, the contribution of the BBR signal at the two wavelengths (1.95 μm and 2.2 μm) is all from superficial tissue in less than 200 μm of depth. Thus, the ratio of the BBR signal from the two wavelengths (at 1.95 μm and 2.2 μm) should be close to the ratio calculated from Planck's equation and reach a stable value at coagulation. Therefore, during the surgical process of heating the ROI, the surgeon can monitor the ratio of BBR from the two wavelengths and can conclude that the tissue is coagulated when the ratio ceases to change substantially despite changes in temperature. That is, the tissue is coagulated when the ratio reaches a steady state and there are only negligible changes in the ratio despite temperature change.

It is also contemplated to generate a virtual thermal map of the temperature within the ROI by extrapolating the temperature measured at a single location (e.g., at the site of the application of thermal energy) and the temperatures at the boundaries of the ROI. This is accomplished by combining a single point temperature measurement with a bioheat transfer simulation. The heat transfer process during tissue heating is governed by Pennes' bioheat transfer equation. For accurate simulation, boundary conditions and tissue-related parameters have to be specified. These conditions may include the tissue surface temperature, tissue optical coefficients, such as scattering or absorption coefficients, and thermal related coefficients, such as heat transfer coefficients. The single location tissue measurement may be used to measure these parameters and be combined with the thermal boundary map and simulation methods to produce an accurate virtual heat map.

For one simulation, the tissue heat transfer coefficient must be known, although this value is different under different situations, such as blood perfusion rate in different organs. The tissue heat transfer coefficient parameter is conventionally obtained through ex vivo tissue study, but using BBR calibration as described above, the tissue heat transfer coefficient may be measured in vivo through fiber thermal sensing. The fundamental idea is based on pulsed photothermal radiometry. Combining BBR and photothermal radiometry, the tissue heat transfer coefficient may be measured in vivo.

Measuring the optical tissue absorption and scattering coefficients of tissue are necessary for simulating light distribution in tissue. Tissue absorption can be measured by measuring optical acoustic effect. Short laser pulses can be absorbed by tissue and then generate an acoustic wave. An optical interferometer, such as an optical coherent tomography (OCT) device, can be employed to detect the sound wave, which has strength that is proportional to tissue absorption coefficients. The OCT device can measure the tissue extinction coefficient, which is the sum of the absorption coefficient and scattering coefficient. As long as the absorption coefficient can be determined, the scattering coefficient can also be discovered.

With the above-measured coefficients and boundary conditions measured as described herein, a virtual heat map within the ROI can be generated during thermotherapy using Pennes bioheat equation. The OCT image can also provide tissue structure images, which can also be used to provide tissue responses during heating.

A simulation to estimate the temperature at various positions relative to the fiber/catheter is contemplated. By measuring and recording temperatures during many surgical procedures, a large database of thermal data may be obtained and then used in similar situations to estimate temperatures when thermal measurements are not available. The simulation produces a thermal map with temperatures determined by measurement of the thermal boundaries in addition to augmented reality images placed thereupon for the surgeon to view. Thus, real images based on measured temperature and virtual images based on data from other surgeries are combined together to create hybrid images in an augmented reality (virtual) thermal map.

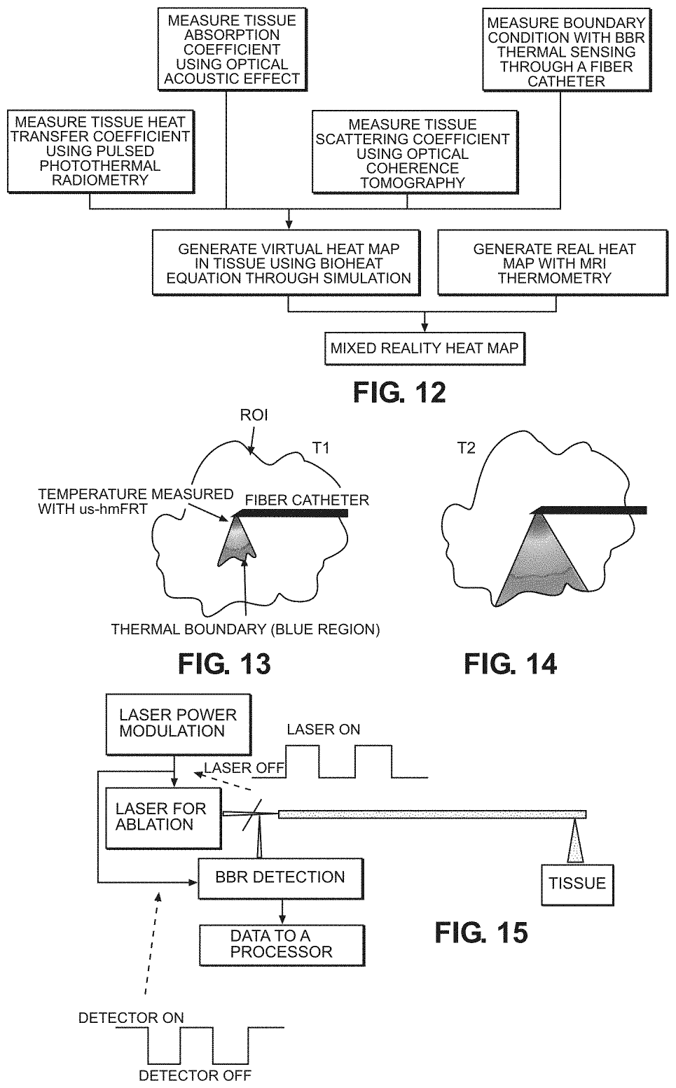

In critical surgery, it is extremely important to map the heat distribution in real-time, such as in brain tumor surgery with interstitial laser therapy, where MRI thermometry is adapted to map heat distribution. However, MRI thermometry has a slow update rate (1 frame/8 secs.). Between frames, surgeons may lose guidance. The virtual heat map can be inserted into the intervals between the MRI generated heat map to continually provide the heat distribution in tissue in real time. Once the measured heat map from the MRI thermometry is updated, the virtual heat map can be compared with the measured heat map. The parameters may be adjusted to match the measured heat map. This method may be referred to as “thermal guidance with mixed reality” and the algorithm flow chart for generating a mixed reality heat map is shown in FIG. 12.

This detailed description in connection with the drawings is intended principally as a description of the presently preferred embodiments of the invention, and is not intended to represent the only form in which the present invention may be constructed or utilized. The description sets forth the designs, functions, means, and methods of implementing the invention in connection with the illustrated embodiments. It is to be understood, however, that the same or equivalent functions and features may be accomplished by different embodiments that are also intended to be encompassed within the spirit and scope of the invention and that various modifications may be adopted without departing from the invention or scope of the following claims.

1. A method of displaying temperature information of living tissue, the method comprising:
(a) inserting a catheter into the living tissue with at least a portion of the catheter penetrating a region of interest of the tissue, the region of interest having boundaries;
(b) conveying thermal energy through the catheter to the region of interest, thereby raising the temperature of the tissue in the region of interest;
(c) detecting blackbody radiation at least at the catheter, and thereby calibrating an ultrasonic thermometry device, by conveying the detected blackbody radiation through the catheter;
(d) measuring tissue temperature using ultrasonic thermometry at least adjacent the boundaries; and
(e) displaying a human-perceivable image representing the boundaries of the region of interest and at least tissue temperature adjacent the boundaries.

2. The method in accordance with claim 1, wherein the step of displaying further comprises combining data from the step of detecting the boundaries and from the step of measuring tissue temperature.

3. The method in accordance with claim 1, further comprising a step of detecting the boundaries of the region of interest.

4. An apparatus for conveying energy to a site and detecting blackbody radiation with a wavelength of less than or equal to about 2.7 μm emanating from the site, the apparatus comprising:
(a) a silica fiber;
(b) means for conveying thermal energy to the site through the fiber; and
(c) means for detecting blackbody radiation from the site.

5. The apparatus in accordance with claim 4, wherein the means for detecting blackbody radiation is configured to detect blackbody radiation in a wavelength range between about 1.4 μm and about 2.7 μm.

6. A combination of a silica optical fiber and living tissue into which the fiber is inserted, the fiber connected to a device that is configured to detect blackbody radiation emanating from the tissue and convey energy to the tissue.

7. The combination in accordance with claim 6, wherein the device is configured to detect blackbody radiation in a wavelength range between about 1.4 μm and about 2.7 μm.

8. A method of determining when living tissue has coagulated, comprising:
(a) measuring a first blackbody radiation signal ratio of the tissue at a first tissue temperature and a first time;
(b) measuring a second blackbody radiation signal ratio of the tissue at a second,
(c) comparing the signal ratios from steps (a) and (b) and calculating a difference between the first signal ratio and the second signal ratio; and
(d) repeating steps (a)-(c) until the difference between the first blackbody radiation signal and the second blackbody radiation signal is negligible.

9. The method in accordance with claim 8, wherein the first and second blackbody radiation signal ratios comprise blackbody radiation detected at a first wavelength and at a second wavelength, wherein water absorbs blackbody radiation less at the second wavelength than the first wavelength.

10. The method in accordance with claim 9, wherein the first wavelength is about 1.95 μm and the second wavelength is about 2.2 μm.
